(19) United States
(12) Patent Application Publication
(10) Pub. No.: US 2017/0326275 A1
(43) Pub. Date: Nov. 16, 2017

(54) Chitosan Hydrogel Microbead

(71) Applicant: KiOmed Pharma, Herstal (BE)

(72) Inventors: Renaud LECLER, FEXHE-LE-HAUT-CLOCHER (BE); Mickaël CHAUSSON, Huy (BE); Pierre DOUETTE, Embourg (BE); Guillem ROCASALBAS, Liege (BE); Sandrine GAUTIER, Liege (BE)

(21) Appl. No.: 15/535,313

(22) PCT Filed: Dec. 7, 2015
(86) PCT No.: PCT/EP2015/078770

§ 371 Date: Jun. 12, 2017

(30) Foreign Application Priority Data
Dec. 12, 2014 (FR) 14 62367

(51) Int. Cl. A61L 27/52; A61L 27/12; A61L 27/20

(52) U.S. Cl. CPC A61L 27/52; A61L 27/20; A61L 27/12; A61L 2400/12; A61L 2400/06

(57) ABSTRACT

The present invention relates to hydrogel microbeads comprising at least water, chitosan, at least one polyphosphate compound, water being present at a concentration of at least 85% by mass of hydrogel, said microbeads having an average number diameter ranging from 100 to 900 μm. The present invention also relates to their manufacturing method and their uses, in particular in pharmaceutical compositions or medical devices, more particularly for treating an articular pathology.

The present invention relates to hydrogel microbeads, to their manufacturing method and to their uses, in particular in pharmaceutical compositions or medical devices, more particularly for the treatment of a joint pathology.

Chitosan is a polymer of natural origin having an interest known for many years. This polysaccharide may be obtained from animal sources like from crustacean shells, but also from fungal sources from cell walls of fungi.

Chitosan is for example used as a matrix for encapsulating active ingredients. It is then generally used as a pharmaceutical carrier. These carriers may appear in solid form, in particular when they are dried or freeze-dried. Many patents relate to this technology.

In parallel with this technology, chitosan-based particles have been developed as hydrogels, of different sizes. These compositions have a particularly high level of water making them very different from the aforementioned matrices. Generally, water represents more than 85%, or even more than 90% by mass of the composition. Such hydrogel particles have a composition and very different properties from the chitosan matrices mentioned above. Such hydrogel particles are notably used in tissue engineering or also as vectors of active compounds.

However, not any hydrogel is suitable for forming beads which may be easily injected into the human or animal body.

From patent EP 2 538 987 B1 (US 20120321678) hydrogel beads are known based on chitosan and alginate, in particular for their uses in intra-articular supplementation. In this patent, the subject is to improve the effects of a hydrogel by combining it with hydrogel beads, which may be used in at least this specific application. Notably these beads should have a long lifetime after injection at the intra-articular level as well as interesting mechanical properties wherein the injection is achieved. Such beads are particularly interesting because of their properties, in particular their elastic properties, provided during the intra-articular injection. The hydrogel beads according to this patent however have trabeculae. The trabeculae are fibril or filamentary species present inside the hydrogel.

Application WO 2009/150651 further describes a hydrogel based on the combination of two chitosan solutions of different characteristics, starting from an acid solution gradually brought to a neutral pH. The first chitosan is highly acetylated with an acetylation level comprised between 40% and 60%. The second chitosan is highly dis-acetylated with an acetylation level of at most 20%. Such hydrogels are sensitive to the temperature which allows them to gel in situ, after injection into the human or animal body. The application does not teach how to form hydrogel particles.

The object of the present invention is to provide hydrogel particles based on chitosan.

The present invention also has the object of providing hydrogel particles based on chitosan for which the concentration is higher than those of certain prior hydrogel beads, having about 0.5% by mass of chitosan relatively to the mass of the hydrogel, while retaining good mechanical properties, in particular for applications in intra-articular injection.

The object of the present invention is further to improve the stability of the chitosan beads.

In particular, the present application has the object of providing hydrogel beads not having any solid particles, like trabeculae.

The present invention also has the object of providing hydrogel beads for which the composition comprising chitosan, optionally mixed with other compounds. This composition is advantageously homogeneous.

From the state of the art it emerges that the preparation of such hydrogel beads is not easy since patent EP 2 538 987 B1 describes the formation of trabeculae and the application WO 2007/13514 recommends the use of a chitosan derivative for overcoming the technical problem of the formation of solid particles.

The further object of the present invention is to provide hydrogel beads for which the manufacturing method may be industrialized, preferably by limiting the manufacturing cost and by ensuring good reproducibility of the thereby manufactured particles.

In particular, the object of the present invention is to simplify the manufacturing method, notably by reducing the variability of the method, a variability which in particular is encountered when several biopolymers are used as chitosans of different nature or mixtures of chitosan and alginate, and further notably by reducing the number of steps of the method and the number of raw materials to be applied.

In particular, the present invention has the object of avoiding the preparation of a preliminary solution containing the alginate, while retaining the ‘hydrogel’ nature of the microbeads, i.e. the capability of remaining stable while retaining a large amount of water, and of remaining deformable and elastic upon compression.

Advantageously, the object of the invention is to provide microbeads having good properties, for example good mechanical properties for the contemplated applications, and more specifically for intra-articular injections, for example good elastic and shock absorption properties, good resistance to crushing, and good adhesion on the tissues.

In this respect, the hydrogel beads according to the prior method described in patent EP 2 538 987 B1 have a large diameter. The article of Oprenyeszk et al., 2013, shows that such particles have a diameter from 600 to 900 micrometers (μm). The hydrogel beads are therefore not suitable for intra-articular injections which require thin needles with a small diameter, notably within the scope of joint diseases treated by intra-articular viscosupplementation and of chronic joint diseases. Such diseases are typically cartilage pathologies, which cause lesions and defects of the cartilage and an articular pain, like arthrosis, for example due either to ageing or to an accident. In order to ensure the ease of injection by the practitioner, increase the comfort of the patient, limit the risks of poorly placed injection and avoid risks of infection, in particular during repeated injections, thin needles should be used.

The present invention therefore has the object of providing an injectable composition via an intra-articular route which facilitates repeated injections, notably through thin needles such as for example needles of 18 to 22 Gauge type as recommended for the most visco-supplementation products presently marketed, in particular above 21 Gauge, or optionally even thinner, for example 23 Gauge. It is therefore desired to develop microbeads for which the properties and the diameter distribution may be adjusted so as to be able to be easily injected through thin needles, in particular microbeads for which the average number diameter is less than 900 μm, preferably less than 800 μm, preferably less than 700 μm, and further preferably for which the D(0.9) is less than 900 μm, preferably less than 800 μm, preferably less than 700 μm.

It was discovered that the technical problems mentioned above may be solved by the present invention. In particular, the invention applies a specific manufacturing method for hydrogel microbeads based on chitosan, which does not comprise the formation of solid trabeculae in the microbeads, thereby avoiding the drawbacks mentioned in the prior art, surprisingly. It is apparent upon reading the prior art discussed above that the solution provided by the present invention was not obvious for one skilled in the art.

Moreover, the microbeads of the invention give the possibility of increasing the proportion of chitosan. For this purpose, the principle of the gelling of the alginate with a calcium ion is not the principle which is applied. Indeed, the invention uses the principle of cross-linking of the chitosan with polyphosphate ions in order to obtain a composition of chitosan hydrogel. The formation of chitosan solutions in the presence of sodium beta-glycerophosphate is already known, for example solutions of chitosan and of beta-glycerophosphate having the property of gelling when the temperature increases, for example at the temperature of the human body. The stabilization of solid and dry microbeads of chitosan is also known by combining it with sodium tripolyphosphate. The formation of complexes based on chitosan and on phytic acid (inositol hexakisphosphate) in solution is further known, or even capsules of millimetric sizes consisting of chitosan and phytic acid. However, the prior art does not preclude the possibility of forming microbeads of chitosan which are in the form of a hydrogel and having suitable properties for having them pass through a needle with a suitable diameter for achieving intra-articular injections, for example with the purpose of treating an articular pathology, such as for example a lesion of the cartilage, in particular in a human being.

The present invention further gives the possibility of increasing the stability of the chitosan by acting on the non-covalent cross-linking of the chitosan, and not on the gelling of the alginate in the presence of a calcium ion, via the chelation of the calcium ion by the alginate.

Notably it was not obvious for one skilled in the art that the association of chitosan with a polyphosphate compound would have given the possibility of obtaining hydrogel beads with good properties, in particular mechanical properties, for an intra-articular injection, by visco-supplementation, and injectable through a thin needle.

The invention more specifically relates to a particle or a hydrogel bead, and more particularly a hydrogel microbead.

Thus, the present invention relates to a hydrogel microbead comprising at least water, chitosan, at least one polyphosphate compound, the water being present at a concentration of at least 85% by mass of the hydrogel, said bead having an average number diameter ranging from 100 to 900 μm.

In particular by <<beads>> is meant a particle with a substantially spherical shape. Such a shape may be appreciated through a microscope. Such a bead may have imperfections inherent to the manufacturing process. By “microbead” is meant a bead having a size of less than 1,000 micrometers.

The beads according to the invention advantageously have an average number diameter comprised between 200 and 800 micrometers, and advantageously comprised between 300 and 700 micrometers.

According to an alternative, the beads according to the invention have an average number diameter preferably comprised between 100 and 500 micrometers, and preferably between 200 and 500 micrometers. The measurement of the average number diameter of the beads according to the invention is preferably achieved with optical microscopy, according to the following method:

The average number diameter, the standard deviation and the variation coefficient of a population of microbeads are measured by means of a reverse optical microscope, for example of the OLYMPUS brand, model CKX41 and provided with a camera and an objective 20X. A fraction of the population of microbeads is placed in an observation cup itself placed under the objective. A digital photograph is taken after adjusting the image. The program “LABSENSE” of the OLYMPUS brand and accompanying the microscope allows plotting of straight lines calibrated according to the used objective. A minimum of 20 beads is selected on the image and a diagonal is plotted by the program on each of them. The program gives the possibility of directly viewing the measurement. These measurements are transferred on a spreadsheet, for example EXCEL of the MICROSOFT brand and the following variables are computed.

The average is calculated by means of the formula:

$$\overline{x} = \frac{1}{n} {\sum }_{i = 1}^{n} {x}_{i}$$

The standard deviation is calculated by means of the formula:

$$s = \sqrt{\frac{1}{n} {\sum }_{i} {( {x}_{i} - \overline{x} )}^{2}}$$

Le variation coefficient Cᵥ is calculated by means of the formula (standard deviation divided by the average):

$${C}_{v} = \frac{s}{\overline{x}}$$

<<n>> represents the number of beads of the sample for which the diameter is measured, <<xᵢ>> represents the measured diameter for a bead, <<i>> being an integer ranging from 1 to n.

The diameter distribution of the microbeads may also be measured by a laser diffraction method, for example by means of a piece of equipment Mastersizer from Malvern, for example a Mastersizer 2000, by using a measurement procedure preferably according to the ISO13320:2009 standard or according to the monograph of the European Pharmacopeia EP2.9.31, applying the theory of Mi.

Advantageously, the bead distribution is narrow, i.e. with a low dispersion of the diameter of the beads relatively to the average diameter, i.e. for example a dispersity index of at least 2 measured by laser diffraction, for example as mentioned before.

Advantageously, the microbeads have a D(0,1) comprised between 100 and 600 micrometers, and advantageously comprised between 200 and 400 micrometers.

According to an alternative, the microbeads have a D(0,5) comprised according to an alternative between 100 and 800 micrometers, and preferably between 300 and 700 micrometers.

Preferably, the microbeads have a D(0,5) comprised according to an alternative between 300 and 600 micrometers.

Advantageously, the microbeads have a D(0,9) comprised between 100 and 1,000 micrometers, and advantageously comprised between 300 and 950 micrometers.

According to a specific alternative, the microbeads have a D(0,9) comprised between 100 and 950 micrometers, and advantageously comprised between 300 and 900 micrometers, and further advantageously between 300 and 800 micrometers.

According to a specific alternative, the microbeads have a D(0,9) comprised between 200 and 700 micrometers, and advantageously comprised between 300 and 650 micrometers.

The invention notably relates to hydrogel beads either sterilized or not, whose D(0.9) diameter is:
less than 700 μm (which may be injected via a 19 Gauge needle),
less than 650 μm (which may be injected via a 20 Gauge needle),
less than 500 μm (which may be injected via a 21 Gauge needle).

D(0,1), D(0,5) and D(0,9) are measured by laser diffraction as aforementioned.

Advantageously, the hydrogel microbeads comprise at least 95% of water by mass. According to a specific alternative, the hydrogel microbeads comprise at least 97% by mass of water.

Advantageously, the chitosan is present at a concentration from 0.3 to 10% by mass, and the polyphosphate compound(s) being present in a sufficient amount in order to form a hydrogel microbead, the mass percentages being expressed with respect to the mass of the microbead.

According to an embodiment, chitosan is present in the hydrogel at a concentration comprised between 0.5 and 5%, and preferably between 1.0 and 3% by mass of chitosan, the mass percentages being expressed based on the mass of the microbead.

The mass percentages expressed relatively to the mass of the hydrogel microbead are expressed relatively to the constituents of the hydrogel. The mass percentages expressed relatively to the mass of the hydrogel microbead are meant preferably of the hydrogel microbead without any active agent, for example cells, polypeptides, proteins, polynucleosides or polynucleotides, which have a therapeutic activity and which are not directly required for the preparation of the hydrogel.

According to a specific alternative, the microbead comprises a chitosan mass between 1.0 and 3.0%, and for example 1.5%.

The chitosan is referenced under the CAS No. 9012-76-4. The chitosan of the invention is a polysaccharide preferably prepared from a fungal source. Preferably it is extracted and purified from secure and abundant biotechnological or food fungal sources such as Agaricus bisporus or Aspergillus niger. The chitosan is obtained by hydrolysis of an extract rich in chitin. Chitin is a polysaccharide consisting of several N-acetyl-D-glucosamine units connected together through a bond of type β (1,4). The chitosan consists of the D-glucosamine units (deacetylated units) and of N-acetyl-D-glucosamine units (acetylated units) connected together through bonds of the β type (1,4) and forms a polymer of the poly(N-acetyl-D-glucosamine)-poly(D-glucosamine) type.

The chitosan of the invention is therefore advantageously of fungal origin, and preferably stemming from the mycelium of a fungus of the Ascomycetes type, and in particular of Aspergillus niger, and/or of a fungus Basidiomycetes, and in particular Lentinula edodes (shiitake) and/or Agaricus bisporus. Preferably the fungus is Agaricus bisporus. Any origin or method for preparing chitosan may be used. A method for preparing chitosan is the one described in the patents stemming from the application WO03068824 (EP1 483 299; U.S. Pat. No. 7,556,946).

The chitosan is advantageously a chitosan which is not chemically modified by a coupling reaction through a covalent bond with one or several other chemical species.

The chitosan is advantageous for its capability of forming particles of three-dimensional structure of the hydrogel type, mechanically resistant, its biocompatibility and its biodegradability after administration or implantation. Further, the presence of chitosan in such hydrogel particles is desired for its intrinsic, physico-chemical and/or biological properties, for example for its capability of adhering to the biological surfaces or its capability of stimulating healing of tissues.

According to an alternative, the average molecular mass of the chitosan is less than or equal to 80,000. According to an alternative, the average molecular mass of the chitosan is comprised between 15,000 and 70,000, and preferably between 35,000 and 60,000.

According to another alternative, the average molecular mass of the chitosan is greater than 80,000. In particular, the average molecular mass of the chitosan is greater than 140,000. The upper limit is generally imposed by the viscosity of the chitosan solution. The use of a chitosan is preferred for which the average molecular mass is less than 1,000,000.

Preferably, here, the average molecular mass is the average viscosity molecular mass (Mv), calculated from the intrinsic viscosity according to the Mark-Houwink equation. The intrinsic viscosity is measured by capillary viscosimetry, with a capillary viscosimeter of the Ubbelohde type, according to the method of the monograph of the European Pharmacopeia EP2.2.9. The flow time of the solution is measured through a suitable capillary tube (Lauda, for example capillary tube Ubbelohde 510 01 of diameter 0.53 mm) by means of an automatic viscosimeter Lauda Visc, first at the initial concentration of chitosan, and then for several dilutions, for example according to the recommendations of the EP2.2.9 method. The reduced intrinsic viscosity is inferred therefrom for each of the concentrations. The reduced viscosity is plotted versus temperature, and the value is extrapolated to the concentration 0 in order to infer therefrom the intrinsic viscosity. For example, the reduced viscosity (η_{red} in ml/g) of the i dilutions should be plotted versus the concentration C of the i dilutions (g/ml) according to formula 5.

[η_{red}]=(t₁−t₀)−(1−C). Formula 2.

In order to calculate the average viscosimetric mass, the Mark-Houwink equation is applied with the constants k and alpha recommended by Rinaudo et al. (Int. J. Biol. Macromol., 1993, 15, 281-285), according to DA of the chitosan, according to one of the three following formulae.

Mv=([η]//0.082)^{(1/0.76)}, for a DA of 2%; Formula 3.

Mv=([η]//0.076)^{(1/0.76)}, for a DA of 10% (for example 11.5%); Formula 4.

Mv=([η]//0.074)^{(1/0.76)}, for a DA of 20% (for example 21%). Formula 5.

For the intermediate DA values, a linear interpolation is achieved for calculating the average viscosimetric mass (Mv).

As an example, the chitosan may have an acetylation degree comprised between 10 and 40%, and preferably between 10 and 25%.

The acetylation degree is determined by potentiometry. The chitosan is dissolved in a solution of hydrochloric acid. The excess of hydrochloric acid which did not react with the amine functions of the chitosan is assayed by a titrated solution of sodium hydroxide. The number of moles of D-glucosamine unit present in the chitosan is thereby calculated and subsequently the acetylation degree, by substraction.

For example, the chitosan solution at a concentration of 1.5% (m/v) in acetic acid 1% (v/v) may have a dynamic viscosity comprised between 100 and 500 mPa·s. Advantageously, the chitosan of the present invention may have a dynamic viscosity comprised between 50 and 400 mPa·s in a 1.5% solution.

The dynamic viscosity of the 1.5% chitosan solution (m/v) is preferably comprised between 150 and 300 mPa·s, and further preferably between 200 and 280 mPa·s. The viscosity is typically measured by viscosimetry with a rotating mobile, for example a Brookfield DV2T device at a speed of rotation of 5 rpm with a <<Spindle SC4-182>> at 25° C.

The polyphosphate compound is preferably a salt of an organic or inorganic polyphosphate compound.

From among the organic polyphosphate compounds, mention may be made for example of phytic acid, in particular sodium inositol hexakisphosphate, or a glycerophosphate, for example sodium beta-glycerophosphate.

From among the inorganic polyphosphate compounds, mention may be made for example of tripolyphosphate, in particular sodium tripolyphosphate.

Advantageously, the polyphosphate compound is selected from among phytic acid, like for example a sodium salt of phytic acid, a tripolyphosphate, such as for example sodium tripolyphosphate, a glycerophosphate like for example a sodium beta-glycerophosphate, and any of their mixtures.

The phytic acid or myo-inositol hexaphosphoric acid or further inositol hexakisphosphoric acid is a biomolecule of raw formula C₆H₁₈O₂₄P₆. Phytic acid is a compound of ubiquitous plants, present at 1-5% by mass of most cereals, nuts, oil from seeds, spores, and pollens. It typically represents a proportion of 60 to 90% of the total phosphorus of the seeds. It is again found in the form of a mixture of salts, typically calcium/magnesium and potassium salts in certain portions of the seeds. This molecule is highly charged with six phosphate groups extending from the center of the myo-ionisol core, each of the groups having its own pKa. The properties of phytic acid may again be found for example in the article of Evans et al. (Titration studies of phytic acid, JAOCS, 59, 4, 1982).

For example, phytic acid is present in the hydrogel of the microbeads at a concentration from 0.5 to 10% by mass. According to a specific alternative, the phytic acid is present in the hydrogel at a concentration of at least 1%, and preferably of at least 2% by mass of phytic acid.

According to a specific alternative, the microbead comprises between 2 and 10%, and more particularly from 4 to 8% by mass phytic acid.

Advantageously, the present invention relates to a microbead comprising from 1 to 2% of chitosan and at least 3% by mass of phytic acid.

An example of composition of the microbead comprises 1.5% of chitosan and 4 to 8% of phytic acid by mass relatively to the mass of the total composition of the microbead.

According to a specific alternative, the microbead comprises between 0.5 to 5%, and more particularly from 1 to 5% by mass of tripolyphosphate, preferably sodium tripolyphosphate.

Advantageously, the present invention relates to a microbead comprising from 1 to 1% of chitosan and at least 0.5% by mass of tripolyphosphate, preferably sodium tripolyphosphate.

An example of a composition of the microbead comprises 1.5% of chitosan and from 1 to 2% of tripolyphosphate, preferably sodium tripolyphosphate, by mass relatively to the mass of the total composition of the microbead.

According to a specific alternative, the microbead comprises between 2 and 10%, and more particularly from 4 to 8% by mass of glycerophosphate, preferably sodium beta-glycerophosphate.

Advantageously, the present invention relates to a microbead comprising from 1 to 2% of chitosan and at least 3% by mass of glycerophosphate, preferably sodium beta-glycerophosphate.

An example of a composition of the microbead comprises 1.5% of chitosan and from 4 to 8% of glycerophosphate, preferably sodium beta-glycerophosphate by mass relatively to the mass of the total composition of the microbead.

Advantageously, the microbead comprises a basic amount sufficient for raising the pH to a value allowing the formation of hydrogel microbeads of the invention.

Among the bases, mention may be made of mineral bases such as for example sodium hydroxide, sodium carbonate, potassium hydroxide, etc.

After washing with successive water baths, the microbead contains water, chitosan and the polyphosphate compound.

In addition to water, the chitosan, the polyphosphate compound, the microbead of the present invention may comprise various excipients and/or various active ingredients.

From among the excipients, mention may be made of agents promoting the gelling of the bead, agents for modifying the viscosity, agents for modifying the flow properties, and agents for modifying the kinetics of degradation of the microbeads in vivo, etc.

The composition of the microbeads is advantageously non-thermogelling. Indeed, in order to ensure good stability of the beads before and after injection, typically via an intra-articular route, the gelling of the sol-gel transition should not occur after or at the moment of the injection. Advantageously, the microbeads of the invention are in the form of a hydrogel before their application, in particular by injection into the body of a subject in need thereof, notably at the intra-joint. In particular, the microbeads are in the form of a hydrogel during their storage, for example at room temperature or under cold conditions, and are not sensitive to variations of temperature between 8 and 40° C., i.e. they are not disintegrated or degraded in this temperature range.

In this respect, the microbeads may be suspended in a viscous solution or a hydrogel, which itself advantageously may be thermogelling. This is typically a hydrogel of one or several gelling polysaccharides. The present invention further relates to a viscous solution or a hydrogel, either thermogelling or not, comprising a plurality of microbeads of the invention.

The composition of the microbead according to the invention may also comprise compounds of interest, most particularly at a pharmaceutical level (pharmaceutical active ingredient) and even more specifically for an intra-articular action, more particularly on the cartilage. These are more specifically beneficial agents in compositions for intra-articular use, for example for reducing pain or reducing inflammation.

From among such agents, mention may be made of anti-inflammatory drugs, more particularly non-steroidal drugs, anesthetic agents, analgesic agents, in particular of the opioid type, corticosteroids, anti-neoplastics, monoclonal antibodies, vitamins, minerals, contrast agents, etc.

Mention may notably be made from among non-steroidal drugs (NSAIDS) for example of: Diclofenac™, Ibuprofen™, Piroxicam™; anesthetics, for example: Lidocaine™, Bupivacaine™; opioid analgesics for example: codeine, morphine; corticosteroids, for example: dexamethasone, prednisone; anti-neoplastic agents, for example: Methotrexate™; antiviral agents, for example: Acyclovir™, Vidarabine™; monoclonal antibodies, for example: Humira™ Infliximab™.

The microbeads of the invention, in particular which may be used as injectable supplements via an intra-articular route may also contain compounds such as cells, proteins, polynucleotides (RNA, DNA), minerals like for example: selenium, strontium, vitamins like for example: tocopherol, or other active ingredients like for example curcumin.

From among the cells, it is of particular interest to use chondrocytes, strain cells, or cells having the capability of producing active substances.

The hydrogel microbeads according to the present invention have a homogeneous hydrogel structure. The hydrogel microbeads according to the present invention do not have any trabeculae, or any solid particles inherent to the preparation of the hydrogel. It is not excluded that active agents are in the solid or colloidal form, for example in a nanoparticle form.

Advantageously, the microbeads of the invention are cohesive. The cohesion of the beads is not lost during friction between hands.

Further, the beads advantageously have a good resistance to compression, in particular during handling between the fingers. Such a resistance may for example be evaluated by the compressive resistance measurement on a mechanical compression bench (for example an Instron Bluehill piece of equipment). It is also possible to use a nano-indentation technique (for example with the range of equipment of brand PI Series Picoindenter marketed by Hysitron or Mach 1 marketed by Biomomentum) or further a suitable tribology technique (for example with the range of equipment of the brand TI Series Triboindenteur marketed by Hysitron).

Further advantageously, the beads are deformable and may be injected with a needle for which the inner diameter is smaller than the maximum diameter of the beads.

This has a great advantage for injection into a tissue since, with equal inner diameter, it is possible to use a needle with a smaller outer diameter, thereby forming a hole of a smaller diameter.

The microbeads may be advantageously manufactured according to the method of the invention.

The invention therefore relates to a method for preparing hydrogel microbeads according to the invention.

More specifically, the method of the invention comprises the addition of a chitosan solution to a cross-linking solution comprising at least one polyphosphate compound, and then the gelling of the chitosan in the presence of the cross-linking solution in order to form the hydrogel microbeads.

The chitosan is preferably dissolved in an aqueous solution with a pH below 7. Such a solution typically comprises an acid, preferably a weak acid, like for example an organic acid. Advantageously it is possible to use acetic acid. The solution typically comprises 0.5 to 3% of acetic acid expressed by volume relatively to the total volume of the chitosan solution.

The pH of the chitosan solution is typically comprised between 2 and 6.5, and preferably comprised between 3.5 and 6.0.

Advantageously, the osmolarity of the chitosan solution at the concentration of 1.5% (m/v) ranges from 100 to 300 mOsm/kg, at the temperature of 25° C.

The determination of the osmolarity of the solutions is carried out with an automatic micro-osmometer (Osmometer Type 15M of the Loser Messtechnik brand). The piece of equipment is calibrated beforehand with a solution of 300 mOsm/kg. The sample is placed in a container provided for this purpose, and is set to the standard measurement temperature.

The gelling of the droplets of the chitosan solution in the form of microbeads occurs under conditions capable for gelling of the chitosan solution and of the polyphosphate compound.

According to an alternative, it is possible to add one or several polyphosphate compounds to the chitosan solution, before having this solution fall dropwise into the cross-linking solution comprising one or several polyphosphate compounds either identical with or different from those added to the chitosan solution. It is thus possible for example to add 0.01 to 1% (m/m), and preferably from 0.1 to 0.5% (m/m, meaning mass/mass), of the polyphosphate compound into the solution of chitosan.

According to an alternative, the cross-linking solution has a pH comprised between 8 and 14, more particularly between 9.5 and 14, adjusted by adding a diluted solution of a base, for example sodium hydroxide.

According to an alternative, the cross-linking solution is a solution of phytic acid and of sodium hydroxide having a pH comprised between 5.5 and 7, for example of about 6.

According to an alternative, the cross-linking solution is a solution of phytic acid and of sodium hydroxide having a pH comprised between 8 and 14.

According to another specific alternative, the cross-linking solution is a solution of phytic acid and of sodium hydroxide having a pH comprised between 8 and 10, for example of about 9.

According to another specific alternative, the cross-linking solution is a solution of phytic acid and of sodium hydroxide having a pH comprised between 11 and 14, for example of about 13.

Advantageously, it is possible to vary the properties of the hydrogel microbeads according to the pH of the cross-linking solution used.

The concentration of the base in the cross-linking solution is determined by one skilled in the art in order to obtain the desired pH. It is typically possible to use a base concentration ranging from 0.01 to 1 M, and more specifically ranging from 0.05 to 0.75M in the cross-linking solution.

Gelling is achieved by putting the chitosan solution dropwise in contact with a cross-linking solution comprising at least one polyphosphate compound, or optionally one or several other gelling agents.

The device for forming the drops and their gelling may be of the <<prilling>> type, i.e. a prilling process. A device is preferably used, comprising a nozzle for producing drops having an internal diameter greater than or equal to 100 μm, for example it is possible to use a diameter of 100 or 150 μm.

For the formation of the microbeads, it is possible to use a batch method, for example by letting the solution pass through a needle by means of a peristaltic pump. It is also possible to use a continuous method, industrially suitable. As an example of a continuous method, mention may for example be made of an electromagnetic method with a laminar jet, also called prilling, like with the continuous equipment VAR-D (marketed by Nisco) or others proceeding like for example a method for electrostatically forming droplets, or with a flow of coaxial air, or a flow of dynamic air, or by gravity, or by nebulization (or “spray drying”), or further by continuous extrusion with the outflowing jet cut by means of a rotary tool (called Jetcutter, like with the Genialab equipment). When the drops of the chitosan solution fall into the cross-linking solution comprising the polyphosphate compound, stirring may be achieved, for example by means of a magnetic bar, for example at a speed comprised between 50 and 500 rpm.

Advantageously, the method comprises the washing of the microbeads, preferably with an aqueous solution, and further preferably with water. Intensive washing is preferably achieved under conditions capable of obtaining hydrogel microbeads with constant diameter from one wash to the other.

The method of the invention for example comprises the recovery of the microbeads by gravity. When the stirring is stopped, the beads settle at the bottom of the container containing the solution, typically the container of the washing bath. The solution for example may be removed with a suction device, like through a needle for which the characteristics (typically the diameter) do not allow passing of the microbeads. After removal of the solution, the microbeads only remain in the container, which may be handled for subsequent operations.

The present invention also relates to sterile microbeads. Advantageously, the microbeads are sterilized with humid heat, typically by using an autoclave. Typically, the autoclave conditions are a temperature of about 121° C., for a period of at least 15 minutes.

Thus, the present invention further relates to a method for sterilizing microbeads described in the invention.

The present invention relates to a pharmaceutical composition or a medical device comprising a plurality of microbeads according to the invention or obtained according to the method of the invention.

The present invention also relates to an injectable pharmaceutical composition comprising the microbeads of the invention.

The present invention relates to a pharmaceutical composition or a medical device consisting in an artificial synovial fluid.

According to an alternative, the pharmaceutical composition or the medical device is useful in the treatment of an articular pathology.

According to an alternative, the pharmaceutical composition or the medical device is useful in the treatment of pain or discomfort associated with a pathology affecting a joint or for slowing down the progression of an articular pathology.

The present invention further relates to a medical device, optionally as one or several packaging kits, optionally physically separated comprising a syringe, a needle and a pharmaceutical composition or medical device according to the invention, said syringe comprising a reservoir optionally pre-filled with the pharmaceutical composition or the aforementioned medical device.

The present invention relates to such a medical device for its use in the treatment of a joint pathology comprising the injection of a pharmaceutical composition or a medical device according to the invention via an intra-articular route.

The present invention also relates to hydrogel microbeads according to the invention, or obtained according to the invention for its use in the treatment of a pathology, by injection into the human or animal body, optionally with the presence of an injectable solution or hydrogel.

For example, the needle used is a needle selected from the 18 to 22 gauge needles, still preferably of 22 gauge and more. For example, the needle used has normal walls, thin or extra-thin.

The present invention further relates to a method for supplementing synovial fluid, in particular during a pathology affecting a joint, such as for example arthrosis (osteoarthritis) or cartilage lesions, etc.

The present invention further relates to a method for combating pain or discomfort associated with a pathology affecting a joint.

The present invention further relates to a method for combating an inflammation of a joint.

More specifically, such pathologies are for example: osteoarthritis (primary (idiopathic) or secondary osteoarthritis), rheumatoid arthritis, injury of a joint (for example trauma or an injury related to repeated joint mobility), a pathology of the cartilage (for example, chondrocalcinosis or chondromalacia), septic arthritis.

The present invention further relates to a method for reducing or combating against the pain associated with a disease, for example like the aforementioned ones, or further for slowing down progression thereof.

The present invention further relates to a method for improving bone repair, in particular cartilage repair.

Such methods typically comprise the intra-articular injection of a composition comprising microbeads according to the invention. According to an alternative, the method according to the invention comprises a plurality of injections. According to an alternative, it is possible to achieve an injection once or twice per month for several months. According to another alternative, two injections are achieved spaced apart by a variable period. According to another alternative, a single injection is achieved.

The methods of the invention are useful for a subject in need thereof, such as for example a subject requiring treatment of a joint pathology.

The injection site(s) is(are) typically selected from among: a knee, a shoulder, a hip, a temporo-mandibular joint, a carpo-metacarpal joint, an elbow, an ankle, a wrist, a joint of the hand, an intervertebral disc or another joint. According to an alternative, the injection is achieved in an articular cavity, in contact with cartilage.

When microbeads are added to a fluid, a viscous solution or a hydrogel, for example to a viscosupplement, for example comprising hyaluronic acid, an increase in the elastic modulus (G′) measured by rheology is obtained. This expresses the resistance to stress of the hydrogel microbeads, and therefore an improvement in the resistance to stress of the fluid containing the microbeads. By extension, the capability of the fluid to absorb impacts, for example when it is injected into a joint and more specifically the joint of the knee, is improved in the presence of beads. The viscosity of the fluid at a physiological temperature is unchanged advantageously, so that it remains easy to inject through a needle with an acceptable diameter by a physician, and that it keeps sufficient viscosity for acting as a viscosupplement.

Thus, the present invention further relates to the use of microbeads according to the invention, or obtained according to the invention, for modifying the properties, in particular the mechanical properties, of a viscosupplement for example injectable at a joint.

More generally, the microbeads of the invention are used in tissue engineering or as vectors of active compounds, in particular as vectors of pharmaceutically active compounds.

The expressions of the type “comprises from . . . to . . . ” “comprised between . . . and . . . ” “ranging from . . . to . . . ” or their equivalents include the mentioned limits, unless indicated otherwise. According to an alternative, the limits of the interval are excluded.

A composition, method or defined process such as in the present invention are covered by <<according to the invention>> or equivalent terms, including according to any of the alternatives, particular or specific embodiments, independently or according to any of their combinations, including according to the preferred characteristics.

In the figures:

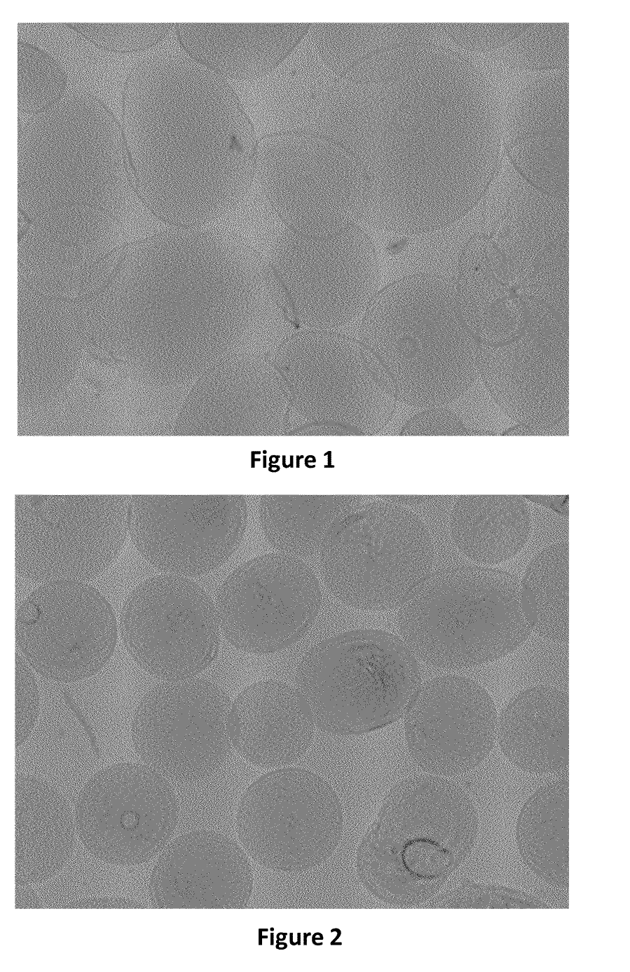

FIG. 1 illustrates an observation by optical microscopy of hydrogel microbeads formed in the presence of the cross-linking solution containing sodium beta-glycerophosphate at the concentration of 5% and sodium hydroxide at the concentration of 0.1M (No. 7).

FIG. 2 represents an observation by optical microscopy of hydrogel microbeads formed in the presence of the cross-linking solution containing sodium tripolyphosphate at the concentration of 2%, the sodium glycerophosphate at the concentration of 5% and sodium hydroxide at the concentration of 0.1M (No. 8).

Other objects, features and advantages of the invention will become clearly apparent to one skilled in the art following the reading of the explanatory description which makes reference to examples which are only given as an illustration and cannot in anyway limit the scope of the invention.

The examples are an integral part of the present invention and any feature appearing to be novel relatively to any prior state of the art from the description taken as a whole, including the examples, is an integral part of the invention in its function and in its generality.

Thus, each example has a general scope.

On the other hand, in the examples, all the percentages are given by mass unless indicated otherwise, and the temperature is expressed in degree Celsius unless indicated otherwise, and the pressure is atmospheric pressure, unless indicated otherwise.

### EXAMPLES

TABLE 1
References for the needle size with view to injecting microbeads
according to the ISO9626 (1991:Amd 1:2001) standard

| | | Outer | Internal diameter (ID) | | |
|---|---|---|---|---|---|
| | | diameter | | | Extra- |
| | | (OD) | Normal/regular | Fine/fine | fine/ultrafine |
| | | (min-max) | walls ID | walls ID | walls ID |
| Source | Gauge | (μm) | (μm) | (μm) | (μm) |
| 1 | 29 | 324-351 | 133 | 190 | — |
| | 27 | 400-420 | 184 | 241 | — |
| | 26 | 440-470 | 232 | 292 | — |
| | 25 | 500-530 | 232 | 292 | — |
| | 22 | 698-730 | 390 | 440 | 522 |
| | 20 | 860-920 | 560 | 635 | 687 |
| | 19 | 1030-1100 | 648 | 750 | 850 |
| | 18 | 1200-1300 | 790 | 910 | 1041 |
| 2 | 23 | ~620 | ~325 | — | — |
| | 21 | ~820 | ~500 | — | — |

1 - “The Gauge system for the medical use” in Anesthesia & Analgesia, 2002; the values are extracted from the ISO9626:1991/Amd 1:2001 standard.
2 - “Does needle size matter ?”, in J. Diab. Sci. Technol. 1, 725, 2007.

The outer diameter (OD) (min-max) designates the tolerance according to the aforementioned standard.

Unless indicated otherwise, the mentioned internal diameter of the needles is with normal/regular walls.

### Example 1—Preparation of the Chitosan Solution

An ultrapure chitosan from a fungal source (Synolyne Pharma, Belgium), with an average viscosimetric molecular mass (Mv) of 180,000 (greater than 140,000) and with an acetylation degree (DA) of 27 mol % (greater than 20 mol %) is dispersed in a solution containing 1% (0.167M) of acetic acid, at a concentration of 1.5% (comprised between 1 and 2%) with magnetic or mechanical stirring. The solution is mixed for a period of 3 hours (1 to 12 hours). The solution is filtered on a filter with a pore diameter of 5 μm. At a concentration of 1.5%, the pH of the chitosan solution is of about 4, its osmolarity at 25° C. is of about 150 mOsm/kg, and its dynamic viscosity is of about 220 mPa·s (measured by viscosimetry with a rotating mobile with a Brookfield equipment, at 5 rpm with the Spindle SC4-18).

It is possible to form droplets starting with this chitosan solution with nozzles of a small diameter up to the smallest size available for the piece of equipment VAR-D (Nisco), i.e. with the 100-μm diameter nozzle.

### Example 2—Preparation of the Cross-Linking Solutions Based on Polyphosphate Compounds

The cross-linking solutions are mixtures of polyphosphate compounds alone or combined, with different concentrations, for which the pH is adjusted in the presence of a base like for example sodium hydroxide, or not. A compound selected from sodium tripolyphosphate (TPP, Sigma), sodium beta-glycerophosphate (GP, Safic Alcan), or phytic acid in the form of a anhydride sodium salt (or anhydrous sodium inositol hexakisphosphate, PA, Sigma) are used as a polyphosphate. The concentrations of the polyphosphates compounds and of the base (NaOH), as well as the pH of the cross-linking solutions are reported in table 2.

TABLE 2
Cross-linking solutions based on polyphosphate compounds

| | | | | Feasibility of the |
|---|---|---|---|---|
| | | | | preparation of stable |
| | | | | hydrogel microbeads |
| No. | Polyphosphate | NaOH | pH | (Examples 3 and 4) |
| 1 | 0 | 0.05M | 12.8 | No |
| 2 | 0 | 0.1M | 13.0 | No |
| 3 | 0 | 0.5M | 13.2 | No |
| 4 | TPP 5% | 0 | 8.6 | No |
| 5 | GP 5% | 0 | 9.3 | No |
| 6a | TPP 5% | 0.1M | 12.9 | Yes |
| 6b | TPP 2.5% | 0.05M | 12.4 | No |
| 6c | TPP 1.25% | 0.075M | 12.7 | Yes |
| 7 | GP 5% | 0.1M | 13.0 | Yes |
| 8a* | TPP 2% and GP 5% | 0M | 8.8 | No |
| 8b** | TPP 2% and GP 5% | 0.05M | 12.5 | Yes |
| 8c | TPP 2% and GP 5% | 0.1M | 12.9 | Yes |
| 9 | PA 5% | 0 | 3.0 | No |
| 10 | PA 5% | 0.1M | 6.0 | Yes |
| 11 | PA 5% | 0.3M | 9.0 | Yes |
| 12 | PA 5% | 0.5M | 13.0 | Yes |
| 13 | PA 2% | 0.1M | 6.0 | No |
| 14 | PA 2% | 0.3M | 9.0 | No |
| 15 | PA 2% | 0.5M | 13.0 | No |

*8a: TPP 2% + GP 5% without NaOH => the beads form but are unstable: they break up after 5 to 10 minutes: the beads are not compliant with the microbeads of the invention;
**8b: TPP 2% + GP 5% + NaOH 0.05M => the beads form and remain stable after one hour in the cross-linking solution: the beads are compliant with the invention.

### Example 3—Preparation of Hydrogel Microbeads of Chitosan by Cross-Linking with Tripolyphosphate (TPP) Alone or Combined with Glycerophosphate (GP)

Droplets are formed initially from the solution of chitosan according to Example 1 with an electromagnetic process with a piece of equipment “Encapsulator VarD (Gen 2) (Nisco, Zürich, Switzerland), equipped with a 150-μm diameter nozzle.

The droplets are immersed in a 50-ml volume of one of the cross-linking solutions according to Example 2 (No. 1 at 8c of Table 1), and are stirred for a period of 3 hours by means of a magnetic bar at a velocity comprised between 100 and 1,000 rpm.

When microbeads form, they are then washed with water (about one liter for each wash) several times consecutively. Slight stirring is achieved with a magnetic bar for about one minute between each wash, at a velocity comprised between 100 and 1,000 rpm. The beads are left to settle between each wash. The beads are finally recovered by gravity. A container is obtained containing a known mass of hydrogel beads, as well as a known mass of water.

It emerges from this example that stable hydrogel beads may be formed in the presence of the cross-linking solutions Nos. 6, 6c, 7 and 8c, i.e. only in the presence of polyphosphate salts TPP and/or GP and of a sufficient amount of NaOH.

The stable hydrogel beads cannot be formed in the presence of NaOH alone (Nos. 1 to 3), in the absence of any polyphosphate. In the presence of TPP and/or GP polyphosphate and in the absence of NaOH, the beads formed are not sufficiently stable from a mechanical point of view and do not withstand successive washes.

The conditions of the cross-linking solution which give the best results in terms of cohesion and of stability of the hydrogel microbeads are the conditions Nos. 6c, 7 and 8c. The characteristics of the thereby obtained hydrogel beads are summarized in Table 3.

The microbeads of the invention do not have any solid trabeculae.

The solid trabeculae of chitosan may be sought by optical microscopy after staining the sample with hematoxylin-eosin. As the eosin is anionic with a tendency of binding onto the chitosan, positively charged.
The beads are stained and observed as entire beads (and free) or in the form of sections made with a microtome or further with a bistoury. The sections are then included in paraffin:
   Attaching the beads and including them in paraffin:
   The beads are incubated for 4 hours at 4° C. in a buffer solution of 100 mM sodium cacodylate and 20 mM CaCl₂ at pH 7.4 and 40 g/L of paraformaldehyde.
   The beads are washed 3× by means of a buffer solution of 100 mM sodium cacodylate and 50 mM BaCl₂ at pH 7.4 in order to prevent their disintegration.
   The beads are then dehydrated by successive passages in the baths with increasing concentration baths of methanol, isopropanol and xylene.
   The beads are then included in the paraffin and the paraffin blocks are cut into lamellas with a thickness of 5 μm by means of a microtome (Leica RM 2145).
   Staining with hematoxylin-eosin:
   In order to be stained, the beads sections are de-paraffinated beforehand and rehydrated with successive baths of xylene, ethanol in decreasing concentration and of demineralized water. The entire beads are directly stained.
   The beads or beads sections are incubated for 15 minutes in a solution of Mayer hematoxylin solution.
   The beads or bead sections are rinsed 2× with water, and then by means of a 2.6% NH₄OH solution.
   The beads or bead sections are incubated in a solution of 0.5% aqueous eosin Y and 0.5% acetic acid.
   The beads or bead sections are then rinsed with water and then dehydrated by means of a bath of increasing concentrations of ethanol and then of xylene.
For observation with an optical microscope (Olympus CKX41), the bead sections are then mounted on slides and lamellas or entire beads are placed in a cup with water.
The observation of the internal frame of the bead(s) with the optical microscope gives the possibility of appreciating the presence or the absence of directly visible trabeculae and exacerbated by staining with eosin. If the observed internal frame is homogeneous and without any filaments emerging from the contrast, the bead is described as not including any solid trabeculae.

TABLE 3
Characteristics of the hydrogel microbeadss formed in the presence of GP and of a
GP/TPP mixture in the presence of NaOH (nozzle with a diameter of 150 μm)

| | Diameter distribution | | |
|---|---|---|---|
| | of the hydrogel beads | | |
| Cross-linking | | By laser | Mechanical aspect and |
| solution | By optical microscopy | diffraction | strength |
| No. 6c | ND | D(0.1) = 190 μm | Good strength |
| TPP 1.25% | | D(0.5) = 490 μm | |
| NaOH 0.075M | | D(0.9) = 740 μm | |
| pH = 12.7 | | | |
| No. 7 | average diameter = 645 μm | ND | Beads not very resistant, |
| GP 5% | min diameter = 370 μm | | elastic, round on average |
| NaOH 0.1M | max diameter = 890 μm | | (FIG. 1) |
| pH = 13.0 | | | |
| No. 8c | average diameter = 560 μm | ND | More resistant beads, less |
| GP 5% | min diameter = 420 μm | | elastic, more round |
| TPP 2% | max diameter = 700 μm | | (FIG. 2) |
| NaOH 0.1M | | | |
| pH = 12.9 | | | |

ND: Not determined

In FIGS. 1 and 2 the substantially spherical shape of the hydrogel microbeads of the invention is observed.

From this example, it is concluded that it is necessary to attain a sufficiently high pH in order to obtain hydrogel beads of good stability and integrity and well elastic with polyphosphates TPP and GP.

It is also concluded that hydrogel beads formed by contact with the solution based on TPP and GP in combination are more stable and elastic than the beads formed starting with TPP or GP alone, with an equivalent basic pH.

It is also possible to produce stable hydrogel microbeads of a smaller size with the 100-μm diameter nozzle.

The microbeads of the invention are injectable through fine needles.

### Example 4: Preparation of the Hydrogel Microbeads of Chitosan with Phytic Acid

Droplets are formed starting with the chitosan solution according to Example 1 by an electromagnetic process with a piece of equipment “Encapsulator VarD (Gen 2) (Nisco, Zürich, Suisse), fitted with a 100-μm diameter nozzle.

The droplets are immersed in a 50 ml volume of one of the cross-linking solutions based on phytic acid (PA) according to Example 2 (Nos. 9 to 15 of Table 1), and are stirred for a period of 3 hours by means of a magnetic bar, at a speed comprised between 100 and 1,000 rpm.

When microbeads form, they are then washed with water (about one liter of each wash) several times consecutively. Slight stirring is achieved with a magnetic bar lasting for about one minute between each wash, at a speed comprised between 100 and 1,000 rpm. The beads are left to settle between each wash. The beads are finally recovered by gravity. A container containing a known mass of hydrogel beads, as well as a known mass of water are obtained.

It emerges from this example that stable hydrogel beads may be formed in the presence of the cross-linking solutions Nos. 10, 11 and 12, i.e. only in the presence of polyphosphate PA and of NaOH simultaneously, both components having to be found in a sufficient amount of each other. The characteristics of the thereby obtained hydrogel beads are summarized in Table 4.

In the presence of phytic acid at a concentration of 5% and in the absence of NaOH, the formed beads are not stable overtime and do not resist to the successive washes with water. When NaOH is added, stable beads are formed as soon as the NaOH concentration is of 0.1M (pH 6.0), unlike the cases of the TPP and GP polyphosphates of Example 3 for which the pH should be higher (for example greater than 12.5) so that the beads are stable.

In the presence of phytic acid at a lower concentration of 2% regardless of the amount of NaOH (from 0.1 to 0.5M), beads are formed but they are not stable.

TABLE 4
Characteristics of the hydrogel microbeads formed
in the presence of phytic acid and of NaOH

| | Diameter distribution | |
|---|---|---|
| | of the hydrogel beads (before | |
| Cross-linking | sterilization) | Mechanical aspect and |
| solution | By optical microscopy | strength of the beads |
| No. 10 | Average diameter = 325 μm | Opaque |
| PA 5% | Min diameter = 260 μm | Soft, deformable, elastic |
| NaOH 0.1M | Max diameter = 360 μm | and tender |
| pH = 6.0 | | |
| No. 11 | Average number diameter | Transparent with an |
| PA 5% | comprised between | opaque core, |
| NaOH 0.3M | 100 and 700 μm | Elastic on average, hard |
| pH = 9.0 | | on average |
| No. 12 | Average number diameter | Very transparent, with a |
| PA 5% | comprised between | very dense core and a |
| NaOH 0.5M | 100 and 700 μm | small diameter, |
| pH = 13.0 | | not very deformable and |
| | | elastic, hard |

Min/max diameter: Smallest/largest diameter measured from among the 20 observed beads.

It is concluded that with a sufficient amount of phytic acid (for example 5%) and in the presence of NaOH, it is possible to form hydrogel microbeads regardless of the NaOH concentration and the pH (above 6.0). On the other hand, the NaOH proportion strongly influences the mechanical aspect and strength of the hydrogel beads. It is thus possible to modulate the properties of the microbeads.

The beads are sterilized with humid heat (autoclave—model SYSTEC DX-23, Wettenberg, Germany). The autoclave parameters are the following: temperature of 121° C., duration of 15 minutes.

The thereby sterilized microbeads have an average number diameter comprised between 100 and 700 μm. Specifically, the beads prepared by means of the cross-linking solution No. 10 have an average number diameter of 200 μm.

They are injectable through fine needles.

### Example 5—Rheological Properties

When the microbeads according to Examples 3 and 4 are added to a fluid, a viscous solution or a hydrogel, for example to a viscosupplement based on hyaluronic acid, an increase in the elastic modulus (G′) is obtained measured by rheology (detail of the method). This expresses the resistance to stress of the hydrogel microbeads, and therefore an improvement in the resistance to stress of the fluid containing the microbeads. By extension, the capability of the fluid of absorbing impacts, for example when it is injected into a joint and more specifically the knee joint, is improved in the presence of beads. The viscosity of the fluid at a physiological temperature is unchanged advantageously, so that it remains easy to inject through a needle with a diameter acceptable by a physician, and it keeps sufficient viscosity for acting as a viscosupplement.

### Example 6—Elasticity of the Microbeads

In order to determine the elastic and absorption properties of the hydrogel microbeads, microbeads—prepared according to Examples 3 and 4 with cross-linking solutions Nos. 6a, 6c, 8b, 8c, 10, 11 and 12 of Example 2 are added to a viscous fluid, for example a solution of hyaluronic acid, at 37° C.

The rheological properties with oscillation of the viscous fluid alone and with the viscous fluid with microbeads are measured, by means of a rotary rheometer with shearing of plates, (ARES G2, TA Instruments). This analysis may give information on the time-dependent change of the variables G′, G″ and tan(δ) on a range of given shearing frequencies, for example 0.1 Hz to 100 rad·s⁻¹, and at a given temperature, for example 37° C., the amplitude is set in constant value, for example 1%.

It is shown that the elastic modulus (G′, also called a storage modulus) of the viscous solution comprising the microbeads is significantly greater than the modulus G′ of the solution of the viscous solution without the microbeads, and this regardless of the composition of the cross-linking solution. The difference in G′ is indicative of the elasticity of the microbeads. The value of the modulus G′ of the fluid is increased significantly in the presence of the microbeads, which indicates that the microbeads impart to the fluid a better capability of resisting to the stress and of absorbing impacts.

In parallel, the dynamic viscosity of the viscous solution is measured with and without the microbeads at 37° C., by means of the same piece of rheometry equipment, with continuous rotation, at increasing speed, over a determined shearing range. It is observed that the dynamic viscosity of the viscous fluid is not modified by addition of the microbeads. It thus remains easily injectable through a needle, and may for example act as a viscosupplement for relieving a joint after injection into the joint of the knee.

For example, a mixture of a viscosupplement (based on hyaluronic acid) available commercially (SynVisc®, Sanofi) with the microbeads prepared according to Example 3 with the cross-linking solution No. 10 (phytic acid 5%, NaOH 0.1M, pH 6) is prepared. The 2 solutions (with and without microbeads) containing the same concentration of hyaluronic acid. The elasticity modulus G′ and the dynamic viscosity of 2 solutions are measured at 37° C., according to the measurement parameters reported in tables 5 and 6.

The results are shown in tables 5 and 6. It emerges from this example that the addition of chitosan and phytic acid microbeads causes an increase in the elasticity modulus of the solution of 40% hyaluronic acid, without modifying its dynamic viscosity.

The microbeads are easily injectable through a needle with variable diameter, and in particular with a needle adapted to the intra-articular injection. The microbeads of the invention recovered after injection substantially retain the same size distribution.

Moreover, the same solution of hyaluronic acid with the microbeads (obtained with the solution No. 10 of table 3) is injectable through needles with variable diameter, for example a needle suitable for intra-articular injection. The beads recovered after injection substantially retain the same size distribution.

TABLE 5
Elasticity modulus G′ of commercial hyaluronic acid,
with and without microbeads (No. 10), at 37° C.

| | G′_{HA} | G′_{HA+MB} | Difference | Difference |
|---|---|---|---|---|
| Oscillation | Without | With | G′_{HA+MB} − | (G′_{HA+MB} − G′_{HA})/ |
| frequency | microbeads | microbeads | G′_{HA} | G′_{HA} × 100 |
| (Hz) | (Pa) | (Pa) | (Pa) | (%) |
| 1 | 25 | 35 | 10 | +40% |
| 10 | 66 | 93 | 27 | +41% |

TABLE 6
Dynamic viscosity of commercial hyaluronic acid with
and without microbeads (No. 10), at 37° C.

| | Dynamic viscosity | Dynamic viscosity |
|---|---|---|
| Shear rate | of HA without | HA with |
| (per second) | microbeads (mPa · s) | microbeads (mPa · s) |
| 1 | 32 | 33 |
| 10 | 3.7 | 3.7 |

HA: hyaluronic acid;
MB: Microbead

### Example 6—Stability of the Beads Upon Storage and Injectability

The hydrogel microbeads prepared by means of the cross-linking solutions Nos. 6a, 6c, 8b, 8c, 10, 11 and 12 of Example 2 are stored at 4° C. in an aqueous solution. After storage duration of 3 and 6 months, their aspect, their average number diameter and their size distribution, measured according to the methods of the description, are unchanged. The microbeads are injectable without any difficulty through a fine needle, and substantially retain their size characteristics after injection.

1. A plurality hydrogel microbeads, the hydrogel comprising water, chitosan, and at least one polyphosphate compound, the water is at a concentration of at least 85% by mass of the hydrogel, said microbeads having an average diameter in the range from 100 to 900 μm.

2. The hydrogel microbeads according to claim 1, wherein the microbeads have a D(0.9) that is in the range from 100 and 950 μm.

3. The hydrogel microbeads according to claim 1, wherein the chitosan is at a concentration in the range from 0.3 to 10% by mass of the hydrogel, and the polyphosphate compound(s) is/are at concentration sufficient for forming the hydrogel microbeads.

4. The hydrogel microbeads according to claim 1, wherein the chitosan is at a concentration in the range from 0.5 to 5% by mass of the hydrogel.

5. The hydrogel microbeads according to claim 1, wherein the polyphosphate compound(s) is/are selected from among phytic acid, tripolyphosphates, glycerophosphates, and combinations thereof.

6. The hydrogel microbeads according to claim 1, wherein the hydrogel microbeads are sterilized.

7. A method for preparing hydrogel microbeads comprising adding a chitosan solution to a cross-linking solution comprising at least one polyphosphate compound, and then gelling the chitosan in the presence of the cross-linking solution to form the hydrogel microbeads, wherein the hydrogel microbeads have a concentration of water that is at least 85% by made of the hydrogen, and wherein the hydrogel microbeads have an average diameter in the range from 100 to 900 μm.

8. (canceled)

9. A pharmaceutical composition or a medical device comprising a plurality of microbeads as defined according to claim 1.

10. The pharmaceutical composition or medical device according to claim 9, wherein it consists in an artificial synovial fluid.

11. A method of treatment of an articular pathology, said method comprising administering to a subject in need thereof the pharmaceutical composition or medical device according to claim 9.

12. The method of claim 11, wherein said method treats pain or discomfort associated with a pathology affecting a joint or for slowing down the progression of an articular pathology.

13. A medical device, optionally as one or several packaging kits, optionally physically separated, the medical device comprising a syringe, a needle and a pharmaceutical composition that comprises a plurality of microbeads as defined according to claim 1, said syringe comprising a reservoir optionally pre-filled with said pharmaceutical composition.

14. The method of claim 11, wherein said method comprises injecting the pharmaceutical composition or medical device according to claim 9 via an intra-articular route.

15. A method for modifying the properties of a viscosupplement, said method comprising adding the plurality of hydrogel microbeads of claim 1 to the viscosupplement of microbeads as defined according to any of.

16. The plurality hydrogel microbeads of claim 1, wherein the concentration of the chitosan is in the range from 1 to 3% by mass of the hydrogel.

17. The plurality hydrogel microbeads of claim 1, wherein the polyphosphate compound is selected from the group consisting of a sodium salt of phytic acid, a sodium tripolyphosphate, a sodium beta-glycerophosphate, and combinations thereof.
